(19) United States
(12) Patent Application Publication
(10) Pub. No.: US 2014/0279341 A1
(43) Pub. Date: Sep. 18, 2014

(54) METHOD AND SYSTEM TO UTILIZE AN INTRA-BODY AREA NETWORK

(71) Applicants: Anurag Bhardwaj, Sunnyvale, CA (US); Robinson Piramuthu, Oakland, CA (US); Neelakantan Sundaresan, Mountain View, CA (US)

(72) Inventors: Anurag Bhardwaj, Sunnyvale, CA (US); Robinson Piramuthu, Oakland, CA (US); Neelakantan Sundaresan, Mountain View, CA (US)

(73) Assignee: eBay Inc., San Jose, CA (US)

(21) Appl. No.: 13/826,100

(22) Filed: Mar. 14, 2013

(51) Int. Cl. A61B 5/00

(52) U.S. Cl. CPC A61B 5/6801; G06Q 40/04
USPC 705/37; 455/426.1

(57) ABSTRACT

An intra-body area network (IBAN) is described. An IBAN comprises a plurality of electronic sensing devices. Each from the plurality of electronic sensing devices is embedded in an item that is wearable by the user. The plurality of sensing devices collect respective different types of information and may communicate with each other, as well as with a smart phone of the user.

### TECHNICAL FIELD

This application relates to the technical fields of software and/or hardware technology and, in one example embodiment, to system and method to utilize an intra-body area network.

### BACKGROUND

Some existing consumer electronics products can be worn by a user and can be designed to collect data associated with the wearer's activities. A device may be configured to collect physiological and movement data of a user. For example, highly accurate, low cost Micro-Electro-Mechanical Systems (MEMS) motion sensor devices, such as accelerometers, have already found their way into wearable sensors to perform basic tasks such as step counting and to monitor overall activity levels.

### BRIEF DESCRIPTION OF DRAWINGS

Embodiments of the present invention are illustrated by way of example and not limitation in the figures of the accompanying drawings, in which like reference numbers indicate similar elements and in which:

FIG. 1 is a diagrammatic representation of a network environment within which example method and system to utilize an intra-body area network may be implemented;

FIG. 2 is block diagram of a system to interact with devices within an intra-body area network, in accordance with one example embodiment;

FIG. 3 is a flow chart of a method to utilize an intra-body area network, in accordance with an example embodiment; and

FIG. 4 is a flow chart of a method to update a profile of a user based on data collected in an intra-body area network, in accordance with an example embodiment; and

FIG. 5 is a diagrammatic representation of an example machine in the form of a computer system within which a set of instructions, for causing the machine to perform any one or more of the methodologies discussed herein, may be executed.

### DETAILED DESCRIPTION

Method and system to provide a so-called intra-body area network are described. In the following description, for purposes of explanation, numerous specific details are set forth in order to provide a thorough understanding of an embodiment of the present invention. It will be evident, however, to one skilled in the art that the present invention may be practiced without these specific details.

As used herein, the term “or” may be construed in either an inclusive or exclusive sense. Similarly, the term “exemplary” is merely to mean an example of something or an exemplar and not necessarily a preferred or ideal means of accomplishing a goal. Additionally, although various exemplary embodiments discussed below may utilize Java-based servers and related environments, the embodiments are given merely for clarity in disclosure. Thus, any type of server environment, including various system architectures, may employ various embodiments of the application-centric resources system and method described herein and is considered as being within a scope of the present invention.

As mentioned above, an electronic device may be designed to be worn by a user and can also be able to collect data associated with the wearer's activities. Wearable electronic sensing devices may be distributed over a person's body, as these devices may be embedded in various wearable items, such as glasses, earrings, shoes, shirts, etc. The data collected by various electronic sensing devices distributed over a user's body, may be analyzed, aggregated, or otherwise processed at a processing computing device, such as, e.g., a server computer system. Based on the analyzed and processed data, the server system may generate and send an electronic communication to the user. A processing computing device may also be a smart phone of a user. The electronic sensing devices positioned over a person's body may be designed to communicate with each other and with the smart phone of the user utilizing Near Field Communication (NFC) or short-wavelength radio transmissions. The collection of electronic sensing devices positioned over a person's body, together with a smart phone of the same user that is often located in close proximity to the user's body, may be termed an intra-body area network (IBAN), as these devices are located in close proximity to each other and can communicate via NFC or short-wavelength radio transmissions, such as Bluetooth®. Bluetooth® is a registered trademark of Bluetooth SIG, Inc.

As mentioned above, the data collected by electronic sensing devices within an IBAN of a user can be provided to or obtained by a processing computing device, such as a server computer system or a smart phone of the user. The collected data may be used at a processing computing device, either by itself or in combination with other data, to generate and send an electronic communication to the user. Such electronic communication may be a message, an image, an alert or some other communication. For example, if combined data from the sensors within the IBAN of a user indicates that the user may be intoxicated, a communication from the processing computing device may be a suggestion that the user should call a taxi. Examples of electronic sensing devices include an array of Electroencephalography (EEG) sensors, which can measure brain waves to predict alertness and emotion. Activity can be detected by gyroscopes on torso and limbs of the person. Audio can be detected through a microphone. If the person's brain activity is high (which may be indicated by gamma or high beta brainwave) and the person's limbs are in motion and the audio stream is detected to be a football match, then the information from multiple sensors can be aggregated and a recommendation may be generated for the user with respect to football-related items for sale or for viewing. In one embodiment, a device that is capable of receiving data from the sensors within an IBAN does not have to listen to all sensors at the same time. If a person is wearing an EEG head-set that is provided within the IBAN of the person, a device that is capable of receiving data from the sensors within the IBAN could just listen to brain waves of the person. When the activity level of brain is high, the device that is capable of receiving data from the sensors within the IBAN could start listening to other sensors within the IBAN to detect what is causing the increased brain activity. Once the activity is detected by correlation of multi-sensor data, proper action may then be taken, such as recommendation of items to the person at that moment or in future. Information about the person is thus learned and accumulated for better advice to that person.

An electronic communication generated based on the data collected by electronic sensing devices within an IBAN may also utilize a profile of the user. A profile of the user may be maintained by a so-called IBAN system executing at a server computer system. The profile may be updated dynamically based on the data collected by electronic sensing devices within the IBAN of the user. For example, data collected from a certain electronic sensing device within the IBAN may indicate that the user performs a cardio-intensive activity (e.g., working out at a gym) at certain time of day. Data collected from another electronic sensing device within the IBAN may indicate a current geographic location of a user. The IBAN system may be configured to generate an alert based on the current data from electronic sensing devices within the IBAN and the profile of the user if, e.g., the user's heart rate is indicative of cardio-vascular activity while the user's geographic location is associated with a work place of the user.

An electronic sensing device may be embedded into or attached to a jacket of the user and be configured to detect any size adjustments the user performs on the jacket. Based on the size adjustment information a jacket size for the user may be determined and stored. In another example, an electronic sensing device may be embedded in a running shoe to detect that the integrity of the sole is below acceptable level, which may trigger the IBAN system to suggest that the user replaces her running shoes. Still further types of electronic sensing devices may be capable of detecting and reporting smells, changes in the movement patterns of the user, changes in the speech patterns of the user, etc.

The profile of a user may include a variety of information, such as, e.g., information about the user's existing wardrobe. The data can include colors, sizes, shapes and types of wardrobe items. This data may be utilized as the user is shopping on-line. As the user requests a search with respect of certain item of closing, the IBAN system may filter the results using the size information, user's favorite brand information, and other information stored in the user's profile. The IBAN system may also be configured to determine colors that would be desirable based on the user's current wardrobe, and filter search results based on color-coordination rules that may also be maintained by the IBAN system. Other data that can be maintained, aggregated and processed by an IBAN system includes information associated with music taste of the user, reading patterns and interests of the user, hobbies of the user, etc.

As mentioned above, an IBAN system may be provided at a server computer system. An IBAN mobile application (IBAN app) may be provided at a smart phone of a user. An IBAN app may be configured to be in communication with various electronic sensing devices within an IBAN and also communicate with a server computer system that hosts an IBAN system. An IBAN app may be configured to generate communications for a user directly in response to data received from the electronic sensing devices within the associated IBAN. In some embodiments, an IBAN app may also be configured to generate communications for a user based on data collected by the electronic sensing devices within the associated IBAN and additional information provided by an IBAN system hosted at a server computer system. Method and system to utilize an intra-body area network may be implemented in the context of a network environment 100 illustrated in FIG. 1.

As shown in FIG. 1, the network environment 100 may include electronic sensing devices 110, 112, and 114, a smart phone 120, and a server system 140. The electronic sensing devices 110, 112, and 114, as well as the smart phone 120 may be positioned over a person's body, may communicate with each other utilizing Near Field Communication (NFC) or short-wavelength radio transmissions, and may form an intra-body area network (IBAN) 116. The communication channels NFC or short-wavelength radio transmissions within the IBAN 116 are designated by the broken curved lines in FIG. 1. As mentioned above, The electronic sensing devices 110, 112, and 114 may be embedded in or attached to items of clothing, shoes, headgear, earrings, glasses, etc., and may collect variety of data, such as, e.g., biometrics of the user, geographic location of the user, visual and environmental surroundings of the user, etc. The smart phone 120 may include an IBAN mobile application (IBAN app) 122 that may be configured to receive or obtain, and also process, collected data from the electronic sensing devices 110, 112, and 114. The IBAN app 122 may also be configured to generate communications to the user associated with IBAN. Such communications may include messages, images, alerts, vibration alarms, etc. The IBAN app 122 may further be configured to communicate with the server system 140, and, specifically, with an IBAN system 144 provided at the server system 140. It will be noted, that the server system 140 may be embodied in one or several physical computing devices.

The electronic sensing devices 110, 112, and 114, as well as the smart phone 120 may also be in communication with the server system 140 via a communications network 130. The communications network 130 may be a public network (e.g., the Internet, a mobile communication network, or any other network capable of communicating digital data). For example, the IBAN app 122 executing at the smart phone 120 may communicate raw or processed data collected by the electronic sensing devices 110, 112, and 114 to IBAN system 144 executing at the server system 140. The IBAN system 144, in turn, may analyze, aggregate, or otherwise process the data collected by the electronic sensing devices 110, 112, and 114 and generate communications for the user associated with IBAN. In one embodiment, the communication generated by the IBAN system 144 may be provided to the user via the smart phone 120.

The server system 140, in one example embodiment, may host an on-line trading platform 142. The on-line trading platform 142 hosted by the server system 140, in one example embodiment, provides a place for buyers and sellers to come together and trade almost anything. In the context of one example on-line trading platform, a seller lists an item—most anything from antiques to cars, books to sporting goods. The seller chooses to either accept only bids for the item (an auction-type listing) or to offer the so-called “Buy It Now” option, which allows buyers to purchase the item right away at a fixed price. In some embodiments, the IBAN system 144 may be integrated with the on-line trading platform 142.

Also shown in FIG. 1 is database 150 that may be used to store profiles of users as profiles 152. The IBAN system 144 may be configured to update profiles stored in the database 150, based on data collected by the electronic sensing devices 110, 112, and 114. Example modules that may be included in the IBAN system 144 and/or in the IBAN app 122 are illustrated in FIG. 2.

FIG. 2 is a block diagram of an example system 200 to utilize an intra-body area network, in accordance with one example embodiment. As shown in FIG. 2, the system 200 includes a data collector 202, an aggregation module 204, and a response module 208. The data collector 202 may be configured to collect data from electronic sensing devices that may be part of an IBAN of a user, such as the electronic sensing devices 110, 112, and 114 illustrated in FIG. 1. As mentioned above, electronic sensing devices 110, 112, and 114 may communicate with each other utilizing Near Field Communication (NFC) or short-wavelength radio transmissions, and be embedded in or attached to items of clothing, shoes, headgear, earrings, glasses, etc., and collect data, such as biometrics of the user, geographic location of the user, visual and environmental surroundings of the user, etc. The aggregator 204 may be configured to aggregate data from the electronic sensing devices, such as the electronic sensing devices 110, 112, and 114 illustrated in FIG. 1. The response module 206 may be configured to generate a communication for the user utilizing the collected and aggregated data. One or both of the data collector 202 and the aggregator 204 may be provided at a smart phone of the user. Either or both of the data collector 202 and the aggregator 204 may be provided at a server computing device. The response module 208 may be configured to generate a communication for the user utilizing the collected and aggregated data from electronic sensing devices, such as the electronic sensing devices 110, 112, and 114 illustrated in FIG. 1.

Also shown in FIG. 2 is a profile generator 206. The profile generator 206 may be configured to update a profile of a user based on the aggregated data. The profile of a user may be stored in the database 150 of FIG. 1 as the profiles 152. In one example embodiment the data collector 202 receives a first data, the first data collected by a first electronic sensing device (e.g., the electronic sensing device 110 of FIG. 1), and the aggregator 204 aggregates the first data with a second data, the second data collected from a second electronic sensing device (e.g., the electronic sensing device 112 of FIG. 1) to generate aggregated data. The profile generator 206 then updates a profile of a user based on the aggregated data. The response module 208 generates a communication for the user, utilizing the collected and aggregated data. The response module 208 may generate the communication for the user utilizing not only the aggregated data, but also the profile of the user. For example, as mentioned above, the first data may be from an electronic sensing device embedded in a jacket and may reflect size adjustment information with respect to the jacket. The profile generator 206 may then update a profile of the user utilizing the size adjustment information.

The system 200 may also include a communications module and a results generator (not shown). The communications module may be configured to receive a request from the user directed to an on-line trading system (e.g., the on-line trading system 142 shown in FIG. 1), such as, e.g., a request to show listings of sale items such as jackets. The results generator may be configured request results in response to the request utilizing the size adjustment information in response to the request. Example operations performed by the system 200 can be described with reference to FIG. 3.

FIG. 3 is a flow chart of a method 300 to utilize an intra-body area network, according to one example embodiment. The method 300 may be performed by processing logic that may comprise hardware (e.g., dedicated logic, programmable logic, microcode, etc.), software (such as run on a general purpose computer system or a dedicated machine), or a combination of both. In one example embodiment, the processing logic resides at the server system 140 of FIG. 1 and, specifically, at the system 200 shown in FIG. 2.

As shown in FIG. 3, the method 300 commences at operation 310, where a plurality of electronic sensing devices that can be embedded in respective items wearable by a person (a user) are provided. As mentioned above, the plurality of electronic sensing devices, together with the smart phone of the user, comprise what may be termed an intra-body area network (IBAN), as an IBAN system executing at a server computer system facilitates communication between the electronic sensing devices and the smart phone of the user (operation 320).

At operation 330, the data collector 202 of FIG. 2 collects data from the plurality of electronic sensing devices (e.g., from the electronic sensing devices 110, 112, and 114 of FIG. 1). At operation 340, the aggregator 204 aggregates collected data to generate aggregated data. At operation 350, the response module 208 generates a communication for the user, utilizing the collected and aggregated data. As mentioned above, such communication may include one or more messages, images, alerts, vibration alarms, etc.

FIG. 4 is a flow chart of a method 400 a method to update a profile of a user based on data collected in an intra-body area network, according to one example embodiment. The method 400 may be performed by processing logic that may comprise hardware (e.g., dedicated logic, programmable logic, microcode, etc.), software (such as run on a general purpose computer system or a dedicated machine), or a combination of both. In one example embodiment, the processing logic resides at the server system 140 of FIG. 1 and, specifically, at the system 200 shown in FIG. 2.

As shown in FIG. 4, the method 400 commences at operation 410, where the data collector 202 of FIG. 2 receives data from an electronic sensing device (e.g., the electronic sensing device 110 of FIG. 1). At operation 420, the aggregator 204 of FIG. 2 aggregates the first data with data collected from another electronic sensing device (e.g., the electronic sensing device 112 of FIG. 2) to generate aggregated data. The profile generator 206 of FIG. 2 updates the profile of the associated user at operation 430, utilizing the aggregated data. For example, the electronic sensing device 110 may be monitoring biometrics of a user indicative of food consumption, and the electronic sensing device 112 may be tracking the geographic location of a user. Data aggregated based on the data collected from these two electronic sensing devices may be used to determine that the user frequently eats at a certain restaurant. The profile of the user may then be updated to indicate that the user likes a certain type of food. The updated profile information may be used to, e.g., send the user a discount coupon for certain type of groceries.

FIG. 5 shows a diagrammatic representation of a machine in the example form of a computer system 500 within which a set of instructions, for causing the machine to perform any one or more of the methodologies discussed herein, may be executed. In alternative embodiments, the machine operates as a stand-alone device or may be connected (e.g., networked) to other machines. In a networked deployment, the machine may operate in the capacity of a server or a client machine in a server-client network environment, or as a peer machine in a peer-to-peer (or distributed) network environment. The machine may be a personal computer (PC), a tablet PC, a set-top box (STB), a Personal Digital Assistant (PDA), a cellular telephone, a web appliance, a network router, switch or bridge, or any machine capable of executing a set of instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein.

The example computer system 500 includes a processor 502 (e.g., a central processing unit (CPU), a graphics processing unit (GPU) or both), a main memory 504 and a static memory 506, which communicate with each other via a bus 504. The computer system 500 may further include a video display unit 510 (e.g., a liquid crystal display (LCD) or a cathode ray tube (CRT)). The computer system 500 also includes an alpha-numeric input device 512 (e.g., a keyboard), a user interface (UI) navigation device 514 (e.g., a cursor control device), a disk drive unit 516, a signal generation device 518 (e.g., a speaker) and a network interface device 520.

The disk drive unit 516 includes a machine-readable medium 522 on which is stored one or more sets of instructions and data structures (e.g., software 524) embodying or utilized by any one or more of the methodologies or functions described herein. The software 524 may also reside, completely or at least partially, within the main memory 504 and/or within the processor 502 during execution thereof by the computer system 500, with the main memory 504 and the processor 502 also constituting machine-readable media.

The software 524 may further be transmitted or received over a network 526 via the network interface device 520 utilizing any one of a number of well-known transfer protocols (e.g., Hyper Text Transfer Protocol (HTTP)).

While the machine-readable medium 522 is shown in an example embodiment to be a single medium, the term “machine-readable medium” should be taken to include a single medium or multiple media (e.g., a centralized or distributed database, and/or associated caches and servers) that store the one or more sets of instructions. The term “machine-readable medium” shall also be taken to include any medium that is capable of storing and encoding a set of instructions for execution by the machine and that cause the machine to perform any one or more of the methodologies of embodiments of the present invention, or that is capable of storing and encoding data structures utilized by or associated with such a set of instructions. The term “machine-readable medium” shall accordingly be taken to include, but not be limited to, solid-state memories, optical and magnetic media. Such media may also include, without limitation, hard disks, floppy disks, flash memory cards, digital video disks, random access memory (RAMs), read only memory (ROMs), and the like.

The embodiments described herein may be implemented in an operating environment comprising software installed on a computer, in hardware, or in a combination of software and hardware. Such embodiments of the inventive subject matter may be referred to herein, individually or collectively, by the term “invention” merely for convenience and without intending to voluntarily limit the scope of this application to any single invention or inventive concept if more than one is, in fact, disclosed.

### Modules, Components and Logic

Certain embodiments are described herein as including logic or a number of components, modules, or mechanisms. Modules may constitute either software modules (e.g., code embodied (1) on a non-transitory machine-readable medium or (2) in a transmission signal) or hardware-implemented modules. A hardware-implemented module is tangible unit capable of performing certain operations and may be configured or arranged in a certain manner. In example embodiments, one or more computer systems (e.g., a standalone, client or server computer system) or one or more processors may be configured by software (e.g., an application or application portion) as a hardware-implemented module that operates to perform certain operations as described herein.

In various embodiments, a hardware-implemented module may be implemented mechanically or electronically. For example, a hardware-implemented module may comprise dedicated circuitry or logic that is permanently configured (e.g., as a special-purpose processor, such as a field programmable gate array (FPGA) or an application-specific integrated circuit (ASIC)) to perform certain operations. A hardware-implemented module may also comprise programmable logic or circuitry (e.g., as encompassed within a general-purpose processor or other programmable processor) that is temporarily configured by software to perform certain operations. It will be appreciated that the decision to implement a hardware-implemented module mechanically, in dedicated and permanently configured circuitry, or in temporarily configured circuitry (e.g., configured by software) may be driven by cost and time considerations.

Accordingly, the term “hardware-implemented module” should be understood to encompass a tangible entity, be that an entity that is physically constructed, permanently configured (e.g., hardwired) or temporarily or transitorily configured (e.g., programmed) to operate in a certain manner and/or to perform certain operations described herein. Considering embodiments in which hardware-implemented modules are temporarily configured (e.g., programmed), each of the hardware-implemented modules need not be configured or instantiated at any one instance in time. For example, where the hardware-implemented modules comprise a general-purpose processor configured using software, the general-purpose processor may be configured as respective different hardware-implemented modules at different times. Software may accordingly configure a processor, for example, to constitute a particular hardware-implemented module at one instance of time and to constitute a different hardware-implemented module at a different instance of time.

Hardware-implemented modules can provide information to, and receive information from, other hardware-implemented modules. Accordingly, the described hardware-implemented modules may be regarded as being communicatively coupled. Where multiple of such hardware-implemented modules exist contemporaneously, communications may be achieved through signal transmission (e.g., over appropriate circuits and buses) that connect the hardware-implemented modules. In embodiments in which multiple hardware-implemented modules are configured or instantiated at different times, communications between such hardware-implemented modules may be achieved, for example, through the storage and retrieval of information in memory structures to which the multiple hardware-implemented modules have access. For example, one hardware-implemented module may perform an operation, and store the output of that operation in a memory device to which it is communicatively coupled. A further hardware-implemented module may then, at a later time, access the memory device to retrieve and process the stored output. Hardware-implemented modules may also initiate communications with input or output devices, and can operate on a resource (e.g., a collection of information).

The various operations of example methods described herein may be performed, at least partially, by one or more processors that are temporarily configured (e.g., by software) or permanently configured to perform the relevant operations. Whether temporarily or permanently configured, such processors may constitute processor-implemented modules that operate to perform one or more operations or functions. The modules referred to herein may, in some example embodiments, comprise processor-implemented modules.

Similarly, the methods described herein may be at least partially processor-implemented. For example, at least some of the operations of a method may be performed by one or processors or processor-implemented modules. The performance of certain of the operations may be distributed among the one or more processors, not only residing within a single machine, but deployed across a number of machines. In some example embodiments, the processor or processors may be located in a single location (e.g., within a home environment, an office environment or as a server farm), while in other embodiments the processors may be distributed across a number of locations.

The one or more processors may also operate to support performance of the relevant operations in a “cloud computing” environment or as a “software as a service” (SaaS). For example, at least some of the operations may be performed by a group of computers (as examples of machines including processors), these operations being accessible via a network (e.g., the Internet) and via one or more appropriate interfaces (e.g., Application Program Interfaces (APIs).)

Thus, method and system to utilize an intra-body area network has been described. Although embodiments have been described with reference to specific example embodiments, it will be evident that various modifications and changes may be made to these embodiments without departing from the broader spirit and scope of the inventive subject matter. Accordingly, the specification and drawings are to be regarded in an illustrative rather than a restrictive sense.

1. A method comprising:
detecting, using at least one processor, a plurality of electronic sensing devices, each from the plurality of electronic sensing devices being embedded in an item wearable by the user, the plurality of sensing devices collecting respective different types of information;
detecting, using at least one processor, communication between devices from the plurality of electronic sensing devices and a smart phone of the user;
collecting, using at least one processor, data from the plurality of electronic sensing devices;
aggregating, using at least one processor, data from the plurality of electronic sensing devices; and
utilizing, using at least one processor, the collected and aggregated data to generate a communication for the user.

2. The method of claim 1, wherein the collecting and aggregating of the data from the plurality of electronic sensing devices is at the smart phone of the user.

3. The method of claim 1, the aggregating of the data from the plurality of electronic sensing devices is at a server computing device.

4. The method of claim 3, comprising:
receiving first data, the first data collected by a first electronic sensing device from the plurality of electronic sensing devices;
aggregating the first data with a second data, the second data collected from a second electronic sensing device from the plurality of electronic sensing devices, to generate aggregated data; and
updating a profile of a user based on the aggregated data.

5. The method of claim 4, comprising utilizing the collected and aggregated data to generate a communication for the user.

6. The method of claim 5, wherein the communication generated for the user is based on the profile of the user.

7. The method of claim 1, comprising:
receiving a first data from a first electronic sensing device from the plurality of electronic sensing devices, the first electronic sensing device embedded in a jacket, the first data reflecting a size adjustment information; and
updating a profile of the user utilizing the size adjustment information.

8. The method of claim 7, comprising:
receiving a request from the user directed to an on-line trading system; and
in response to the request, generating request results utilizing the size adjustment information.

9. The method of claim 1, wherein the collecting of the data from the plurality of electronic sensing devices is without user interaction.

10. The method of claim 1, wherein the plurality of electronic sensing devices communicate using short-wavelength radio transmissions.

11. A system comprising:
a plurality of electronic sensing devices, each from the plurality of electronic sensing devices being embedded in an item wearable by the user, the plurality of sensing devices collecting respective different types of information, devices from the plurality of electronic sensing devices being in communication with each other;
one or more processors coupled to a memory;
a data collector to collect data from the plurality of electronic sensing devices, using the one or more processors;
an aggregator to aggregate data from the plurality of electronic sensing devices, using the one or more processors; and
a response module to generate, using the one or more processors, a communication for the user utilizing the collected and aggregated data.

12. The system of claim 11, wherein the data collector and the aggregator are provided at a smart phone of the user.

13. The system of claim 11, wherein the aggregator is provided at a server computing device.

14. The system of claim 13, comprising a profile generator, wherein:
the data collector is to receive a first data, the first data collected by a first electronic sensing device from the plurality of electronic sensing devices;
the aggregator is to aggregate the first data with a second data, the second data collected from a second electronic sensing device from the plurality of electronic sensing devices, to generate aggregated data; and
the profile generator is to update a profile of a user based on the aggregated data.

15. The system of claim 14, comprising a response module to generate a communication for the user, utilizing the collected and aggregated data.

16. The system of claim 15, wherein the communication generated for the user is based on the profile of the user.

17. The system of claim 11, wherein:
the data collector is to receive a first data from a first electronic sensing device from the plurality of electronic sensing devices, the first electronic sensing device embedded in a jacket, the first data reflecting a size adjustment information; and
the profile generator is to update a profile of the user utilizing the size adjustment information.

18. The system of claim 17, comprising:
a communications module to receive a request from the user directed to an on-line trading system; and
a results generator to request results, utilizing the size adjustment information in response to the request.

19. The system of claim 11, wherein the plurality of electronic sensing devices communicate using short-wavelength radio transmissions.

20. A machine-readable non-transitory storage medium having instruction data to cause a machine to:
detect a plurality of electronic sensing devices, each from the plurality of electronic sensing devices being embedded in an item wearable by the user, the plurality of sensing devices collecting respective different types of information;
detect communication between devices from the plurality of electronic sensing devices;
collect data from the plurality of electronic sensing devices;
aggregate data from the plurality of electronic sensing devices; and
utilize the collected and aggregated data to generate a communication for the user.
